(19) United States
(12) Patent Application Publication
(10) Pub. No.: US 2002/0192293 A1
(43) Pub. Date: Dec. 19, 2002

(54) Method for manufacture of extended release dosage form

(76) Inventors: Srinivas R. Gadiraju, Randolph, NJ (US); Qing Ha, Lansdale, PA (US); Agber Ifan, Hillsborough, NJ (US); Sau-Hung Spence Leung, Parisippany, NJ (US); Adam Sabouni, Sudbury, MA (US)

Correspondence Address: Mehdi Ganjeizadeh, Warner-Lambert Company, 2800 Plymouth Road, Ann Arbor, MI, 48105, US

(21) Appl. No.: 10/117,742

(22) Filed: Apr. 5, 2002

Related U.S. Application Data
(60) Provisional application No. 60/282,207, filed on Apr. 6, 2001.

(51) Int. Cl. A61K 9/14; A61K 9/50; B29C 47/88; A61K 31/4166

(52) U.S. Cl. 424/499; 514/389; 264/211.12

(57) ABSTRACT

A process for the dry granulation and manufacture of a pharmaceutical formulation comprises the steps of adding sodium phenytoin to a vessel of a mixer/granulator and adding at least one excipient to the vessel. Heat is then applied from the vessel to the sodium phenytoin and excipient to achieve the desired target temperature. Next, while the heat is applied from the vessel, the sodium phenytoin and excipient are mixed and chopped in the vessel to form a granulation suitable for encapsulating into a dosage form. Preferably, the excipients include magnesium stearate, sugar, lactose monohydrate, and talc. In an alternative embodiment, talc is added after chopping and blended into the formulation to form a blend suitable for encapsulating into a dosage form.

This application claims priority from U.S. Ser. No. 60/282,207, the entire contents of which are herein incorporated by reference.

### FIELD OF THE INVENTION

The present invention pertains to a method of manufacturing an extended release dosage form. In particular, the present invention pertains to a method of manufacturing an extended release dosage form of the active pharmaceutical ingredient sodium phenytoin.

### BACKGROUND OF THE INVENTION

In the pharmaceutical development art, a sustained release dosage form may be defined as a preparation which releases a drug, in vivo, at a considerably slower rate than is the case from an equivalent dose of a conventional (nonsustained release) dosage form. The objective of employing a sustained release product is to obtain a satisfactory drug response while at the same time, reducing the frequency of administration. An example of a drug which is popularly used in a sustained release form is chlorpheniramine maleate. In conventional form, the drug may be given as 4-mg doses every four (4) hours or in sustained release form as 1 dose of 12 mg every twelve (12) hours.

Sustained release compositions for the sequential or timed release of medicaments are well-known in the art. Generally, such compositions contain medicament particles, normally administered in divided doses two (2) or three (3) times daily, mixed with or covered by a coating material which is resistant to degradation or disintegration in the stomach and/or in the intestine for a selected period of time. Release of the medicament may occur by leeching, erosion, rupture, diffusion, or similar actions depending upon the nature and thickness of the coating material.

It is known that different pharmaceutical preparations of the same active ingredient will result in different bioavailabilities of the active ingredient to the mammal. Bioavailability or biological availability may be defined as the percentage of the drug liberated from the dosage form administered that becomes available in the body for biological effect. Different formulations of the same drug can vary in bioavailability to a clinically relevant extent and variation may even occur between batches of the same product due to subtle variations in manufacturing procedures.

Many drugs that are usually administered in tablet or capsule form have a low solubility in biological fluids. For many drugs of low solubility, there is considerable evidence that the dissolution rate partially or completely controls the rate of absorption. Bioavailability can also be affected by a number of factors such as the amounts and types of adjuvants used, the granulation process, compression forces (in tablet manufacturing), the surface area available for dissolution, and environmental factors such as agitation in the stomach and the presence of food. Due to these numerous factors, specific formulations play an important role in the preparation of prolonged action solid dosage forms. One disease that typically requires prolonged action solid dosage forms is epilepsy.

Epilepsy is an ancient disease which affects about 1% of the global population. Despite the progress made in antiepileptic drug therapy, there are still many patients who continue to suffer from uncontrolled seizures and medication toxicity. At present, only four (4) major antiepileptic drugs are in use: phenobarbital, sodium phenytoin, carbamazepine, and valproic acid.

Pharmacological activity, in general, and antiepileptic activity in particular, correlate better with a concentration of the drug in the blood (or in some other biophase) than with the administered dose. This phenomenon is due, in part, to variability in drug absorption and disposition between and within individuals, particularly when the drug is given orally. Optimizing drug therapy aims at achieving and maintaining therapeutic and safe drug concentrations in the patient's plasma. It would thus be advantageous that the patient receive a once- or twice-daily dosage regimen.

Phenytoin, 5,5-diphenyl-2,4-imidazolidinedione, is a well-known pharmaceutical agent having anticonvulsant and antiepileptic activity. Due to phenytoin's poor solubility in water, sodium phenytoin, which is much more soluble, is employed in the preparation of injectable solutions of the drug and in solid enteral dosage forms.

Sodium phenytoin has the following formula:

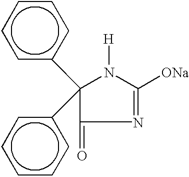

While phenytoin is the antiepileptic drug of choice for many types of epileptic seizures, therapeutic drug monitoring is required because of the difficulty in maintaining an effective therapeutic plasma level of between 10 and 20 μg/mL. In addition to the problems of narrow therapeutic plasma levels, phenytoin has exhibited great variations in bioavailability following its oral administration to patients because of its poor water solubility.

With even the new approaches to phenytoin delivery (ie, Parke-Davis' Dilantin® Kapseals®, which are 100-mg extended sodium phenytoin capsules), it is still necessary for patients to take the drug several times a day to maintain an effective therapeutic plasma level without side effects. With Kapseals, product in vivo performance is characterized by a slow and extended rate of absorption with peak blood concentrations expected in 4 to 12 hours.

While many techniques and processes have been attempted to provide a reliable dosage form of phenytoin comparable to the Dilantin Kapseals, none have been found to be completely satisfactory. Karakasa et al., Biol. Pharm. Bull., 1994;17(3):432-436 in an article entitled “Sustained Release of Phenytoin Following the Oral Administration of Sodium phenytoin/Ethylcellulose Microcapsules in Human Subjects and Rabbits,” studied the release patterns of phenytoin as the sodium salt in combination with ethylcellulose. The sodium phenytoin microcapsules were prepared by mixing 80 weight % of the sodium phenytoin in a 10% (by weight) ethylcellulose solution in ethylacetate. The suspension was stirred, and n-pentane was added dropwise until a phase separation occurred and the microcapsules were obtained. The microcapsules were collected on filter paper, dried and stored. Karakasa et al., point out that following the oral administration of sodium phenytoin, the salt might be easily transferred into free-phenytoin in the acidic fluids of the stomach. As free-phenytoin is practically insoluble in water, its absorption might be incomplete in the gastrointestinal tract. On the other hand, while passing through the stomach, the volume of water penetrating into the ethylcellulose microcapsules might be minimal. Thus, most of the sodium phenytoin in the microcapsules might not be converted into free-phenytoin.

A review article by Boxenbaum in Drug Development & Industrial Pharmacy, 1982;8(v): 1-25, entitled “Physiological and Phamacokinetic Factors Affecting Performance of Sustained Release Dosage Forms” actually suggests that sustained release formulations for drugs such as phenytoin are unnecessary. Boxenbaum points out that dosing schedules of once a day versus three times daily produce similar plasma curves. This results from both the slow absorption and the low solubility of the drug.

Slow release, delayed release, prolonged release, or sustained release phenytoin is a desirable objective. Controlled release oral dosage forms of drugs with long half lives, such as phenytoin, have been disregarded for sustained release formulation since they produce little change in the blood concentration after multiple doses have been administered. The existence of such products can, however, be justified, on the basis of their ability to minimize toxicity and the occurrence of adverse reactions and as providing greater patient convenience and thus, better patient compliance.

Bialer in an article entitled, “Pharmacokinetic Evaluation of Sustained Release Formulations of Antiepileptic Drugs . . . Clinical Implications” in Clinical Pharmacokinetics, 1991;22(1):11-21, also suggests that phenytoin is not a suitable candidate for sustained release formulations. What Bialer and Boxenbaum have failed to realize is that through the novel use of the physical properties of sodium phenytoin and drugs like sodium phenytoin, one can prepare a sustained release formulation that is beneficial to the patient.

A paper by Bourgeois entitled “Important Pharmacokinetic Properties of Antiepileptic Drugs” in Epilepsia, 1995;36(Suppl. 5), discusses the important pharmacokinetic properties of antiepileptic drugs. The author states that a drug's rate of absorption profile is described by its absorption constant (k_{abs}). A high absorption constant results in early and high peak serum concentrations. A high (k_{abs}) value also results in greater fluctuations in drug levels compared with the steadier concentrations resulting from lower (k_{abs}) values. A lower absorption constant can often be produced by formulating an otherwise rapidly absorbed drug in a slow release preparation. However, using enteric coated preparations as part of the process of manufacturing a dosage form does not alter a drug's (k_{abs}) value; they merely delay absorption. An enteric coating is designed to prevent absorption in the acidic environment of the stomach. Consider for example, a patient who has received a single dose of enteric coated valproate. For the first few hours after dosing, serum measurements will fail to detect any drug in the blood. Not until the tablet reaches the alkaline environment of the duodenum does the serum concentration rapidly increase, ultimately achieving a profile similar to that of an uncoated preparation of valproate. Therefore, the enteric coating merely shifts the time concentration profile to the right.

From a review of the prior art, it is evident that a need still remains for a process that can readily and consistently produce a sustained release dosage form for drugs with pH dependent solubilities, such as sodium phenytoin, which provides initial therapeutic levels of the drug and delays the delivery of another fraction of the drug to eliminate excess concentrations for about 1 to 5 hours.

### SUMMARY OF THE INVENTION

The present invention meets the unfulfilled needs described above by providing a process for readily producing a formulation that has a given proportion of a required dose. When sodium phenytoin is the active pharmaceutical ingredient, the formulation exhibits bioequivalency to Dilantin Kapseals dosage forms. Specifically, the present invention comprises the use of a dry granulation process and controlled heat application to form consistent drug granules having a predictable dissolution profile. The process also produces a reliable and consistent releasing matrix of sodium phenytoin. Therefore, standard application of this process provides a reliable manufacturing process of sodium phenytoin dosage forms as well as assuring consistent product performance.

In general, the present invention provides a process for the dry granulation and manufacture of a pharmaceutical formulation wherein the active ingredient is sodium phenytoin. The process comprises the steps of adding sodium phenytoin to a vessel or bowl of a mixer/granulator and adding at least one excipient to the vessel. Next, heat is applied to the sodium phenytoin and the excipient in an amount sufficient to enhance the interaction between the sodium phenytoin and excipient. The constituents are both mixed and chopped in the vessel while the temperature is increased to the desirable level. The resultant granulation is then formed into the desired dosage form, such as capsules.

In an embodiment of the invention, the excipients include magnesium stearate, sugar, and lactose monohydrate, and the process includes the steps of blending talc with the granulation. Alternatively, the talc may be included as one of the excipients initially mixed with the sodium phenytoin in the vessel.

It is believed that patients will benefit from such a formulation since many drugs, like sodium phenytoin, have narrow therapeutic windows which require multiple (three or more) daily dosings.

It is to be understood that both the foregoing general description and the following detailed description are exemplary, but are not restrictive, of the invention.

### BRIEF DESCRIPTION OF THE DRAWING

The invention is best understood from the following detailed description when read in connection with the accompanying drawing, in which

FIG. 1 is a graphical representation showing the effects of target temperature during mixing on dissolution performance;

FIG. 2 is a graphical representation showing the dissolution profile of two sodium phenytoin formulations produced by the process of the present invention compared to the dissolution profile of a Dilantin Kapseals dosage form.

### DETAILED DESCRIPTION OF THE INVENTION

The present invention comprises a dry granulation process with controlled heat application to a mixture of an active pharmaceutical ingredient and one or more excipients to form granules with consistent characteristics. In particular, the present invention is a process for the production of granules of the active pharmaceutical ingredient sodium phenytoin.

The process of the present invention involves the use of a dry granulation device having controlled heating and insulation capabilities. A jacketed mixer/granulator can be used as the dry granulation device, eg, a Collete Mixer/Granulator (Gral®), commercially available from Collete GEI of Belgium. The basic jacketed mixer/granulator design is a high shear mixer/granulator which allows the processing, granulation, and, optionally, drying of the mixture in a totally contained environment. It has the mixing flexibility of both an agitator (low shear) and a chopper (high shear).

The mixer/granulator for use in the present process may have a capacity from about 10 L to about 1200 L. The mixer/granulator for use in the present invention may also have a liquid addition system, a jacketed/insulated vessel, and heating and cooling units. Preferably, and as described above, the mixer/granulator is a jacketed/insulated vessel with heating and cooling units.

The process of the present invention relates to the discovery that therapeutic agents, such as sodium phenytoin, can be processed to yield a dosage form providing sustained blood plasma concentrations of the therapeutic agent. It will be understood by the skilled artisan that the effective amounts are over an intended delivery time and for a desired blood plasma concentration.

It has been found that the controlled application of heat to a mixture of an active pharmaceutical ingredient and at least one excipient during a dry granulation process yields a product that is relatively easy to produce yet exhibits sustained release properties in a reproducible manner. Further, in the case of sodium phenytoin, the product is bioequivalent to commercial Dilantin Kapseals. More specifically, it is believed that by using a jacketed high shear powder mixer/granulator, both mixing and heating can be accurately controlled. This processing feature provides the means necessary to develop a reproducible and validatable process for the manufacture of sodium phenytoin dosage forms.

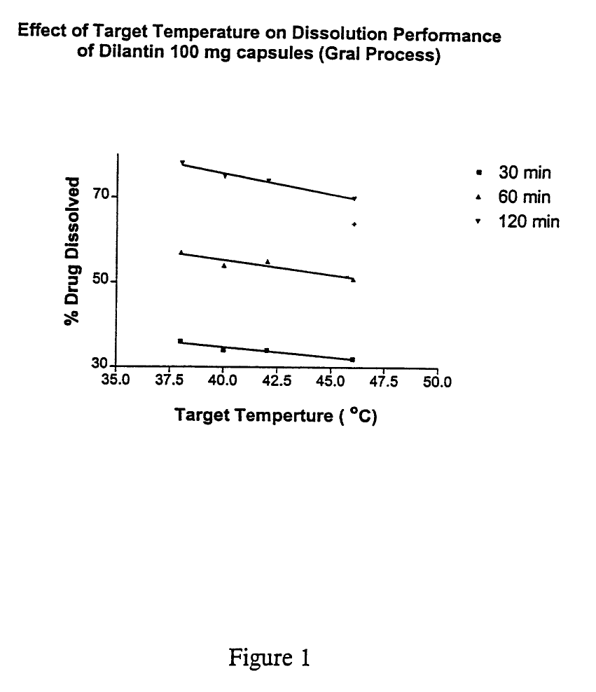

Without ascribing to a particular theory of active pharmaceutical ingredient release, it is believed that the heating of the active pharmaceutical ingredient and the excipient yields granules having an enhanced coating over at least a portion of the active pharmaceutical ingredient. Specifically, it is believed that increasing the mixing temperature facilitates the coating of excipient over a portion of the active pharmaceutical ingredient. This coating provides the desired drug dissolution profile by inhibiting drug dissolution of the coated portion, thereby altering the overall dissolution and diffusion characteristics of the dosage form. It is postulated that an increase in product temperature during the mixing and chopping steps of dosage form production increases this interaction between sodium phenytoin and one or more excipients, typically magnesium stearate. The increase in interaction between the active pharmaceutical ingredient and excipient affects the in situ formation of an extended release matrix. In other words, it is believed that one or more of the excipients form a dissolution inhibiting coating around the active ingredient upon exposure to heat in the mixing vessel of the mixer/granulator. The higher the bulk temperature, the greater the amount of coating around the active ingredient and therefore the greater reduction in solubility of the granules which make up the dosage form. In particular, it was found that the greater the amount of heat (bulk temperature) applied to the active ingredient and excipients during the mixing and chopping steps (described below), the slower the rate of drug release. More specifically, and as shown in FIG. 1, the percent of drug dissolved over time decreases as the sustained temperature of the ingredients contained in the mixing vessel increases. Thus, product temperature control during processing is critical to ensure reproducible dissolution performance.

To achieve the objective of the present invention, an active pharmaceutical ingredient is deposited in the vessel of the mixer/granulator. Preferably, sodium phenytoin is the active pharmaceutical ingredient. Typically, the active pharmaceutical ingredient is added to about 10% to 50% of the overall weight of the mixture to be granulated. (Unless otherwise indicated herein, the percentages of the constituents shall mean weight percentages.) Preferably, 15% to 45% is added to the vessel.

Next, excipients such as fillers and lubricants are deposited in the vessel of the mixer/granulator with the active pharmaceutical ingredient, although the order of addition is not important and may be reversed. Multiple lubricants may be added to the mixture and are well-known in the art, and stearic acid and magnesium stearate are the preferred lubricants, with magnesium stearate most preferred. The lubricant may be added in amounts of about 0.5% to about 5% of the overall weight of the mixture, preferably 1.5% to 4%.

The present invention may also contain at least one filler as an excipient. Suitable fillers are well-known in the art and typically comprise microcrystalline cellulose, sorbitol, mannitol, confectioner's sugar, glucose, lactose monohydrate, and talc. Preferably, confectioner's sugar is added to about 1% to 50% of the overall weight of the mixture, preferably about 10% to 45% of the overall weight of the mixture, more preferably 20% to 40% of the overall weight of the mixture. Talc is added to about 0.5% to 10% of the overall weight of the mixture, preferably about 1% to 5% of the overall weight of the mixture, more preferably about 2% to 4.5% of the overall weight of the mixture. Lactose monohydrate is added to about 1% to about 50% of the overall weight of the mixture, preferably about 10% to 45% of the overall weight of the mixture, more preferably about 20% to 45% of the overall weight of the mixture. Although talc may be added to the vessel with the other fillers, talc may alternatively be added to the mixture just prior to an additional blending step to act as a lubricant. However, if talc is added with the other fillers, the subsequent blending step is unnecessary.

The vessel is heated after all ingredients are added to the vessel or during the subsequent mixing and chopping steps described below. However, it is preferable to heat the vessel to the desired temperature during the mixing step (also described below) after the desired fillers and active ingredients are added to the bowl. Typically, the contents of the vessel may be heated from about 25° C. to 70° C., by indirect heating from the jacketed vessel of the mixer/granulator. Preferably, and in accordance with the present invention, the vessel is heated to about 30° C. to 50° C. Most preferably, the contents are heated to about 32° C. to 40° C. As described above, it is believed the heating step yields granules having an enhanced coating over a portion of the active pharmaceutical ingredient, thereby reducing the solubility of the granule proportionately to the amount of heating applied to the bowl of the mixer/granulator.

To provide homogeneity of the mixture, the temperature is increased to a target temperature level while the mixture is mixed at a speed of about 0.5 to 115 revolutions per minute for a period of about 0.1 to 2 hours. Preferably, the mixture is mixed for about 50 minutes at about 115 revolutions per minute.

Further, while the temperature is increased according to the target temperature level, the mixture is chopped at speeds of about 0.5 to 2400 revolutions per minute for a period of about 0.1 to 2 hours. Preferably, the mixture is chopped for about 15 minutes at about 2400 revolutions per minute.

The two steps of mixing and chopping provide a well-mixed granulation of the ingredients having the desired homogeneity.

Although it is preferred that the temperature of the mixture in the bowl remain substantially uniform throughout the mixing and chopping steps, the temperature of the mixture may be varied from about 25° C. to 70° C. at any time during the process to alter the release profile of the granules and accordingly the dosage form derived from the granules.

As described above, talc may be added to the granules just prior to blending in one embodiment. However, if talc is not added with the other ingredients prior to mixing and chopping, the resultant granulation is subsequently transferred to a blender and blended with talc provided in the amounts detailed above.

Typical blenders for use in the present invention have a volume of about 10 to 75 cubic feet. One such blender which may be used in the present invention is a Patterson Kelley blender. Preferably, the blender has a volume of about 75 cubic feet. The granulation is deposited in the blender and blended for about 5 to 60 minutes at a speed of about 5 to 30 revolutions per minute. Preferably, the granulation is blended 10 minutes at about 10 revolutions per minute.

The resultant blend is subsequently formed in a known manner into the desired dosage form, such as capsules using standard encapsulation procedures. Specifically, a encapsulation machine such as a Hechler and Koch encapsulation machine may be used to encapsulate the formulation into gelatin capsules. Granules may be filled into the body of the capsule dosage form by tamping or dosing, and the capsule may be subsequently sealed using a cap.

### EXAMPLE 1

As described below, two separate formulations were processed in accordance with the present invention. In order to achieve the necessary release profile of sodium phenytoin from a dosage form, the following steps were followed:

First, the ingredients were provided in the amounts set forth in Table 1.

TABLE 1
Formulation

| Ingredients | 100 mg Formulation | 30 mg Formulation |
|---|---|---|
| Sodium Phenytoin, USP | 43.5% | 15.8% |
| Magnesium Stearate, NF | 3.9% | 2.0% |
| Confectioner's Sugar, NF | 24.9% | 39.0% |
| Talc, USP | 2.7% | 4.2% |
| Lactose Monohydrate, NF | 25% | 39% |

Second, all of the ingredients except talc were deposited into the vessel of a GEI-Collete Gral® jacketed mixer/granulator and subsequently dry mixed, chopped and heated, as described in Table 2. Although both the 100-mg formulation and the 30-mg formulation were different compositions, the subsequent mixing and blending steps were the same for both formulations.

TABLE 2
Dry Mixing and Heating Step

| Equipment: | 1200 L GEI-Collette Mixer/Granulator |
|---|---|
| Mixer Speed | 115 rpm |
| Mixing Time | 50 min |
| Chopper Speed | 2400 rpm |
| Chopping Time | 15 minutes |
| Target Bulk Temperature | 40° C. |

To complete the process, each formulation was combined with the talc and subjected to a final blending step in a Patterson Kelley® blender, as described in Table 3.

TABLE 3
Blending Step

| Equipment: | 75 cu ft Patterson Kelley Blender |
|---|---|
| Blending Speed | 10 rpm |
| Blending Time | 10 minutes |

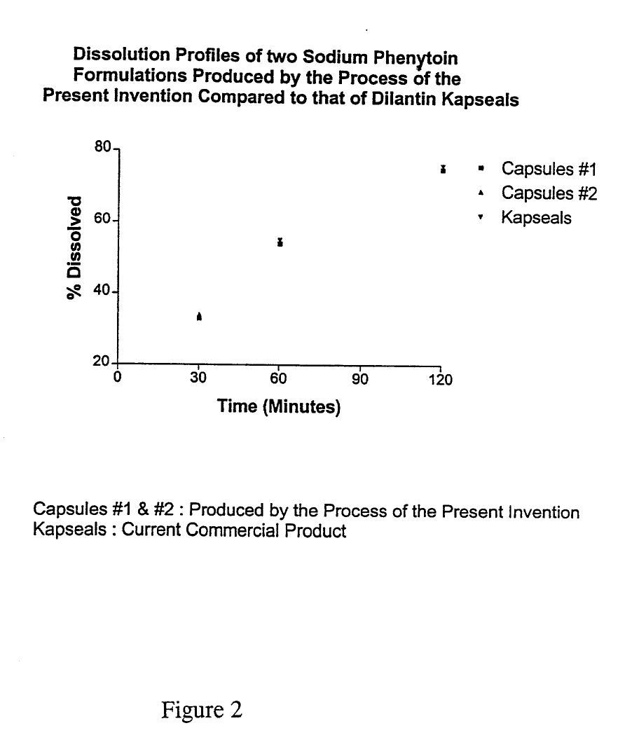

Lastly, the formulation was processed into a standard dosage form through well-established encapsulating procedures discussed above. As shown in FIG. 2, the present invention provides a granulation that upon formation into a dosage form has a similar dissolution profile relative to Dilantin Kapseals.

### EXAMPLE 2

Table 4 provides dissolution data of capsules manufactured from granules produced by the process of the present invention. A USP dissolution test was used for each of the sodium phenytoin formulations. Specifically, each capsule was placed in 900 mL of water, which was maintained at 37° C.±0.5° C. and stirred at 50 revolutions per minute. Samples were collected at 30, 60, and 120 minutes and tested for the amount of sodium phenytoin dissolved.

The capsules were produced in the manner as set forth in Example 1, except that the target temperature was varied as shown in Table 4 and two different sizes of final blenders were used as shown. Table 4 indicates that the granulation of the present invention, when encapsulated into a dosage form, provides a variable release profile which is dependent on temperature and is comparable to that of Dilantin Kapseals.

TABLE 4
Biobatch Selection Experiments

| Product | | Dissolution (%) (SD) | | |
|---|---|---|---|---|
| Temp | Blender Used For | N = 12 | | |
| (° C.) | Final Blending | 30 Minutes | 60 Minutes | 120 Minutes |
| 32 | 2 ft³ PK | 43 (1.8) | 71 (2.2) | 85 (2.3) |
| | 38 ft³ Bin | 47 (2.0) | 73 (2.7) | 89 (2.0) |
| 34 | 2 ft³ PK | 44 (2.9) | 74 (3.2) | 86 (2.3) |
| | 38 ft³ Bin | 43 (2.2) | 72 (3.4) | 85 (2.9) |
| 36 | 2 ft³ PK | 38 (4.3) | 64 (6.8) | 81 (7.6) |
| | 38 ft³ Bin | 38 (2.9) | 62 (2.5) | 80 (1.8) |
| 38 | 2 ft³ PK | 35 (1.3) | 59 (2.8) | 79 (2.2) |
| | 38 ft³ Bin | 36 (2.0) | 58 (2.1) | 79 (1.7) |

### EXAMPLE 3

Table 5 provides stability data of capsules manufactured from granules produced by the process of the present invention. The capsules were produced in the manner as set forth in Example 1. Table 5 indicates that the granulation of the present invention, when encapsulated into a dosage form, exhibits commercially acceptable storage stability over time. In Table 5, “n” stands for the number of samples, and the average percent dissolved is provided in each block with a range of the percent dissolved of all samples.

TABLE 5
Dissolution Stability of a Representative Batch of Sodium
Phenytoin 100-mg Capsules at 30° C./60% Relative Humidity

| | Dissolution (%) (range) | | |
|---|---|---|---|
| Time Interval | 30 Minutes | 60 Minutes | 120 Minutes |
| 0 Month n = 12 | 35 (32-38) | 58 (54-62) | 78 (76-81) |
| 3 Month n = 12 | 35 (34-37) | 59 (57-60) | 78 (77-80) |
| 8 Month n = 6 | 36 (34-37) | 60 (59-62) | 79 (77-81) |

Although illustrated and described herein with reference to certain specific embodiments and examples, the present invention is nevertheless not intended to be limited to the details shown. Rather, the claims should be read to include various modifications within the scope and range of equivalents of the claims, without departing from the spirit of the invention.

What is claimed is:

1. A process for manufacturing a pharmaceutical formulation comprising the steps of:
adding sodium phenytoin to a vessel;
adding at least one excipient to said vessel;
heating said sodium phenytoin and said excipient;
mixing said sodium phenytoin and said excipient to form a mixture; and
chopping said mixture to form a granulation.

2. The process according to claim 1, wherein said vessel further comprises an insulating jacket therearound for improving the uniformity of heat applied to said sodium phenytoin and said excipient.

3. The process according to claim 1, wherein said sodium phenytoin is added to said vessel in an amount of 10% to 50% of the total weight of said granulation.

4. The process according to claim 1, wherein said excipient is selected from the group consisting of at least one of stearic acid, magnesium stearate, microcrystalline cellulose, sorbitol, mannitol, sugar, glucose, lactose monohydrate, and talc.

5. The process according to claim 1, wherein said excipient comprises magnesium stearate.

6. The process according to claim 1, wherein said at least one excipient comprises magnesium stearate, sugar, lactose monohydrate, and talc.

7. The process according to claim 6, wherein said excipient is added to about 50% to 90% of the total weight of said granulation.

8. The process according to claim 6, wherein said magnesium stearate is added to 0.5% to 5% of the total weight of said granulation.

9. The process according to claim 6, wherein said sugar is added to 1% to 50% of the total weight of said granulation.

10. The process according to claim 6, wherein talc is added in an amount of 0.5% to 10% of the total weight of said granulation.

11. The process according to claim 6, wherein said lactose monohydrate is added to 1% to 50% of the total weight of said granulation.

12. The process according to claim 1, wherein the step of heating comprises heating said sodium phenytoin and said excipient to 25° C. to 70° C.

13. The process according to claim 12, wherein the step of heating comprises heating said sodium phenytoin and said excipient to 32° C. to 40° C.

14. The process according to claim 1, wherein the step of mixing comprises mixing at a speed of 0.5 to 115 revolutions per minute.

15. The process according to claim 14, wherein the step of mixing comprises mixing at a speed of 75 revolutions per minute.

16. The process according to claim 14, wherein the step of mixing comprises mixing said mixture for 0.1 to 2 hours.

17. The process according to claim 16, wherein the step of mixing comprises mixing said mixture for 50 minutes at a speed of 115 revolutions per minute.

18. The process according to claim 1, wherein the step of chopping said mixture comprises chopping at a speed of 0.5 to 2400 revolutions per minute.

19. The process according to claim 18, wherein the step of chopping said mixture comprises chopping for a period of 0.1 to 2 hours.

20. The process according to claim 19, wherein the step of chopping said mixture comprises chopping for 15 minutes at 2400 revolutions per minute.

21. The process according to claim 1 further comprising the step of forming said granulation into a dosage form by encapsulating a portion of said granulation.

22. The process according to claim 1 wherein said at least one excipient comprises magnesium stearate, sugar, and lactose monohydrate, and said process further comprises blending said granulation with talc to form a blend.

23. The process according to claim 22 further comprising the step of forming said blend into a dosage form by encapsulating a portion of said blend.

24. A process for the dry granulation and manufacture of a pharmaceutical formulation, the method comprising the steps of:
adding sodium phenytoin to a vessel having an insulating jacket;
adding at least one excipient to said vessel, wherein said excipient is selected from the group consisting of stearic acid, magnesium stearate, microcrystalline cellulose, sorbitol, mannitol, sugar, glucose, and lactose monohydrate;
heating said sodium phenytoin and said excipient to 30° C. to 70° C.;
mixing said sodium phenytoin and said excipient for about 0.1 to 2 hours and at speed of about 0.5 to 115 revolutions per minute to form a mixture;
chopping said mixture at a speed of 0.5 to 2400 revolutions per minute for a period of 0.1 to 2 hours;
adding talc to said mixture; and
blending said mixture to form a blend.

25. The process according to claim 24, wherein said sodium phenytoin is added to said vessel in an amount of 10% to 50% of the total weight of said blend.

26. The process according to claim 24, wherein said excipient comprises magnesium stearate, sugar, and lactose monohydrate.

27. The process according to claim 26, wherein said magnesium stearate is added to 0.5% to 5% of the total weight of said blend.

28. The process according to claim 26, wherein said sugar is added to 1% to 50% of the total weight of said blend.

29. The process according to claim 26, wherein said talc is added to 0.5% to 10% of the total weight of said blend.

30. The process according to claim 26, wherein said lactose monohydrate is added to 1% to 50% of the total weight of said blend.

31. A process for the dry granulation and manufacture of a pharmaceutical formulation, the method comprising the steps of:
adding sodium phenytoin to a vessel having an insulating jacket;
adding at least one excipient to said vessel, wherein said excipient is selected from the group consisting of stearic acid, magnesium stearate, microcrystalline cellulose, sorbitol, mannitol, sugar, glucose, lactose monohydrate, and talc;
heating said sodium phenytoin and said excipient to 30° C. to 70° C.;
mixing said sodium phenytoin and said excipient for about 0.1 to 2 hours and at speed of about 0.5 to 115 revolutions per minute to form a mixture; and
chopping said mixture at a speed of 0.5 to 2400 revolutions per minute for a period of 0.1 to 2 hours to form a granulation.

32. The process according to claim 31, wherein said sodium phenytoin is added to said vessel in an amount of 10% to 50% of the total weight of said granulation.

33. The process according to claim 31, wherein said excipient comprises magnesium stearate, sugar, lactose monohydrate, and talc.

34. The process according to claim 33, wherein said magnesium stearate is added to 0.5% to 5% of the total weight of said granulation.

35. The process according to claim 33, wherein said sugar is added to 1% to 50% of the total weight of said granulation.

36. The process according to claim 33, wherein said talc is added to 0.5% to 10% of the total weight of said granulation.

37. The process according to claim 33, wherein said lactose monohydrate is added to 1% to 50% of the total weight of said granulation.
